(19) United States
(12) Patent Application Publication
(10) Pub. No.: US 2022/0073989 A1
(43) Pub. Date: Mar. 10, 2022

(54) Optimizing Detection of Transplant Injury by Donor-Derived Cell-Free DNA

(71) Applicant: The Regents of the University of California, Oakland, CA (US)

(72) Inventors: Minnie Sarwal, San Francisco, CA (US); Tara Sigdel, San Francisco, CA (US)

(73) Assignee: The Regents of the University of California, Oakland, CA (US)

(21) Appl. No.: 17/416,510

(22) PCT Filed: Dec. 16, 2019
(86) PCT No.: PCT/US2019/067646

§ 371 Date: Jun. 20, 2021

Related U.S. Application Data
(60) Provisional application No. 62/783,009, filed on Dec. 20, 2018.

(51) Int. Cl. C12Q 1/6883

(52) U.S. Cl. CPC C12Q 1/6883; C12Q 2600/16; C12Q 2600/156; C12Q 2600/112

(57) ABSTRACT

Herein are novel methods of detecting subacute and active rejection in graft recipients, including kidney recipients by the measurement of donor-derived cell-free DNA. By the methods, active rejection processes encompassing T-cell mediated rejection may be detected. Also provided herein are novel threshold values for the determination of active rejection that enable higher sensitivity and specificity than prior methods. Additionally, by donor-derived cell-free DNA, subacute rejection processes can be detected, including borderline rejection and other graft injuries.

### CROSS-REFERENCE TO RELATED APPLICATIONS

This application is a 35 USC § 371 national stage application of PCT International Patent Application Number PCT/US2019/067646, entitled “Optimizing Detection of Transplant Injury by Donor-Derived Cell-Free DNA,” filed Dec. 19, 2019, which claims the benefit of priority to U.S. Provisional Application Ser. No. 62/783,009, entitled “Optimizing Detection of Transplant Injury by Donor-Derived Cell-Free DNA,” filed Dec. 20, 2018; the contents which applications are hereby incorporated by reference.

### STATEMENT REGARDING FEDERALLY SPONSORED RESEARCH OR DEVELOPMENT

Not applicable.

### BACKGROUND OF THE INVENTION

Organ transplant procedures have saved countless lives. For example, there are currently approximately 190,000 living kidney recipients in the U.S. However, graft rejection is a serious problem and graft recipients must be monitored for rejection processes. Precision medicine and personalized tailoring of immunosuppressive drug regimens can improve the current state of organ transplant management. Transplantation injuries may be delayed in detection, and therefore treated ineffectively, because diagnosis can be difficult and biopsy, an invasive and potentially toxic procedure, may be inconclusive. Though advances in immunosuppressive drugs, organ procurement methods, and human leukocyte antigen-typing has lowered the number of clinical- and biopsy-confirmed rejection episodes, sub-clinical rejection of kidney grafts remains a significant risk. Kidney transplant management is particularly challenging owing to the lack of sensitivity and specificity of the serum creatinine assay, which, in addition to the late detection of transplant injuries, makes immunosuppression dosage and adjustment far from personalized. Therefore, rapid and non-invasive detection and prediction of allograft injury/rejection holds promise for improving the post-transplantation management of patients who have received kidney allografts.

Diagnosis of renal transplant rejection is generally dependent on an increase in serum creatinine levels or its algorithmic derivative, estimated glomerular filtration rate (eGFR), which indicates altered renal filtration functioning. Since there are many causes of the baseline drift in altered renal filtering in these patients, biopsy is required for definitive diagnosis. Methods of estimating kidney rejection in allograft recipients based on CR or eGFR lack sufficient accuracy. However, biopsies are invasive, morbid and, potentially, costly procedures, which limit their use in clinical practice. Furthermore, biopsy results are often plagued by expert reader variance and can lead to delayed diagnosis of active rejection, after which irreversible organ damage may have occurred. There is a current unmet need for a rapid, accurate, and noninvasive approach to detecting allograft rejection and/or injury-one which may require integration of the current “gold” standard morphological assessments with modern molecular diagnostic tools.

Donor-derived cell-free DNA (dd-cfDNA) detected in the blood of transplant recipients has been reported as a noninvasive marker to diagnose allograft injury/rejection, and holds promise for producing faster and more quantitative results compared with current diagnostic options. Recently, it was demonstrated that plasma dd-cfDNA fraction, typically between 0.3% and 1.2% in stable patients, can discriminate active rejection status from stable organ function in kidney transplant recipients, for example, as describe in Knight et al., Donor-specific Cell-Free DNA as a Biomarker in Solid Organ, Transplantation. A Systematic Review. Transplantation 2018 and in Bloom et al. Cell-Free DNA and Active Rejection in Kidney Allografts. J. Am. Soc. Nephrol. 2017, 28, 2221-2232.

While dd-cfDNA provides a potential diagnostic tool for assessing active rejection, there is a need for improved methods of applying this measure for clinical uses, with higher sensitivity and specificity. Likewise, there remains a need in the art for diagnostic methods to detect other types of rejection injuries. These conditions include borderline rejection, a diagnosis characterized by certain characteristics of acute rejection, but considered to be less than acute rejection. Another category of sub-acute graft injury includes graft injuries due to factors such as drug toxicity or viral infection which do not arise to the level of active rejection. These subacute conditions have significant medical consequences and dictate different treatment options than acute rejection. Accordingly, there is a need in the art for methods of detecting subacute rejection processes in graft recipients. Likewise, there is also a need in the art for methods of treating subacute rejection processes, wherein effective treatment requires accurate detection of subacute rejection processes and the ability to distinguish between acute and subacute processes as they occur.

### SUMMARY OF THE INVENTION

Previously, it was demonstrated that a single nucleotide polymorphism (SNP)-based cell-free assay targeting greater than 10,000 loci worked as a successful screening tool for the detection of fetal chromosomal abnormalities, for example, as described in Dar et al., Clinical experience and follow-up with large scale single-nucleotide polymorphism-based noninvasive prenatal aneuploidy testing. Am. J. Obstet. Gynecol. 2014, 211, e1-e17 and in Ryan et al., Validation of an Enhanced Version of a Single-Nucleotide Polymorphism-Based Noninvasive Prenatal Test for Detection of Fetal Aneuploidies. Fetal Diagn. Ther. 2016, 40, 219-223. Herein is demonstrated the application of a similar approach targeting thousands of SNPs to assess donor cfDNA burden in different transplant rejection injuries over time

The various methods disclosed herein provide the art with a means of detecting subacute rejection processes in transplant recipients. In a first aspect, the scope of the invention encompasses a diagnostic analysis for detecting the occurrence of subacute rejection processes by the use of dd-cfDNA. The novel use of dd-cfDNA for detecting subacute rejection processes advantageously provides the art with a non-invasive, rapid, inexpensive, and effective means of detecting processes that currently cannot be assessed without invasive biopsies. In another aspect, the scope of the invention encompasses improved new dd-cfDNA thresholds for determination of active rejection status, including the occurrence of both T-Cell mediated rejection (TCMR) and antibody mediated rejection (ABMR). In another aspect, the scope of the invention encompasses methods of treatment for active and subacute rejection events, wherein dd-cfDNA assessment is integral to selection of the treatment process.

### BRIEF DESCRIPTION OF THE DRAWINGS

FIG. 1A and FIG. 1B depict discrimination of active rejection by dd-cfDNA (FIG. 1A) versus eGFR (FIG. 1B). Boxes indicate interquartile range (25th to 75th percentile); horizontal lines in boxes represent medians; dots indicate outliers >1.5 times the upper quartile value. p-values for dd-cfDNA and eGFR adjusted using Kruskal-Wallis rank sum test followed by Dunn multiple comparison tests with Holm correction. *** indicates adj. p<0.0001 from all other group comparisons. AR, active rejection; BL, borderline; OI, other injury; STA, stable; dd-cfDNA, donor-derived cell-free DNA; eGFR, estimated glomerular filtration rate.

FIG. 2A and FIG. 2B present predictive statistics for active rejection versus non-rejection predicted by dd-cfDNA (FIG. 2A) versus eGFR (FIG. 2B).

FIG. 3A and FIG. 3B depict discrimination of active rejection by dd-cfDNA in biopsy-matched samples stratified by biopsy type. FIG. 3A: protocol biopsy statistics. FIG. 3B: for-cause biopsy statistics. Boxes indicate inter-quartile range, horizontal lines represent medians. AR, active rejection; BL, borderline; OI, other injury; STA, stable.

FIG. 4 depicts dd-cfDNA as a function of antibody-mediated-versus T-cell—mediated rejection. Boxes indicate interquartile range (25th to 75th percentile); horizontal lines in boxes represent medians; dots indicate all individual data points. p-values for dd-cfDNA adjusted using Kruskal-Wallis rank sum test.

FIGS. 5A and 5B present Table 1.

FIG. 6 presents Table 2.

### DETAILED DESCRIPTION OF THE INVENTION

The scope of the invention encompasses various methods of assessing graft rejection status in a graft recipient by means of dd-cfDNA measurement. In a general implementation, the methods of the invention comprises the steps of
obtaining a sample from a graft recipient;
assaying the sample to determine dd-cfDNA abundance in the graft recipient; and
determining the rejection status of the graft by the measured dd-cfDNA value and an established relationship between dd-cfDNA values and graft rejection status.

The various elements of this process are described next, followed by specific applications for the detection of subacute rejection status, active rejection status, and further diagnostic measures to identify specific types of rejection and/or treat rejection events.

Grafts and Recipients. The methods of the invention are applied for the determination of graft rejection status in a graft recipient. The graft may comprise any selected graft type, for example, a type selected from the group consisting of an organ, tissue, cells, kidney, heart, lung, liver, skin, cornea, intestine, pancreas, limb, digit, bone, ligament, cartilage, and tendon. References to a graft, as used herein will encompass whole organs and portions thereof. In a primary embodiment, the graft is a kidney graft, i.e. a whole or partial kidney.

Transplant is understood to occur between a donor individual and a recipient individual. The transplant donor and transplant recipients may be humans, for example, in some embodiments the recipient may be a human patient in need of treatment for graft rejection. In alternative embodiments, the donor and recipient subjects may comprise non-human animals, for example veterinary patients or test animals. For convenience, the description provided herein will be directed to human subjects. In some embodiments, the donor is a relative of the recipient. In some embodiments, the genetic features of the donor are unknown.

Graft Rejection Status. Graft rejection status, as used herein, may fall within various categories. A first graft rejection status is “stable.” As used herein, a stable rejection status, or stable graft, indicates that the graft is healthy and functioning properly and that there is no substantial immune response against the graft.

A second graft rejection status is “active rejection.” Active rejection, as used herein, means the graft is under substantial attack from the recipient's immune system and/or is undergoing, or is in danger of undergoing, impaired function, failure, necrosis, and other pathologies associated with rejection. Active rejection may encompass various rejection processes, including ABMR, TCMR, and mixed or simultaneous ABMR-TCMR processes, wherein some degree of ABMR and TCMR is occurring simultaneously. Active rejection typically requires augmentation of ongoing treatment or application of additional treatment to reverse the rejection and preserve the graft.

A third graft rejection status is “Subacute Rejection.” Subacute rejection, as used herein, indicates that the graft is undergoing injury, stress, or other pathological processes falling short of, or being different from, the pathologies of active rejection.

In one aspect, the graft type is kidney and the subacute rejection may comprise Borderline Rejection (BL). Borderline rejection may comprise symptoms of as determined by the Banff classification of renal allograft pathology, as known in the art. Borderline rejection may also comprise symptoms as determined under the Cooperative Clinical Trials in Transplantation (CCTT) classification system, as known in the art.

Subacute rejection may also encompass what will be referred to herein as “Other Rejection” (OR). OR encompasses any non-borderline impairment or injury of the graft short of or different from active rejection processes. OR may encompass immune-mediated injury, infection, or drug toxicity effects. In one embodiment, OR includes Early acute rejection without any clinical graft dysfunction-also called subclinical AR, which is often only picked up by protocol biopsy. In one embodiment, OR encompasses viral infection, for example, infection by a polyomavirus family member, for example, a BK virus or BKV.

Dd-cfDNA Analysis. The methods of the invention encompass the assessment of dd-cfDNA in a sample derived from a graft recipient. Donor-derived cell-free DNA, in the context of a graft recipient, is DNA derived from graft cells found in the graft recipient, for example, circulating in the blood of the recipient. Dd-cfDNA is produced by the death and lysis of cells within the graft. When the graft is under attack from the recipient's immune system or is undergoing other injury or dysfunction, the amount of circulating dd-cfDNA increases. Dd-cfDNA may be assessed in a sample derived from the recipient, for example, in serum obtained from a blood sample obtained from the recipient. The selected sample type may comprise any type, or mixture of types, of biological material wherein the dd-cfDNA derived from graft injury is present. Exemplary samples include blood, serum, tissue, including graft tissue, interstitial fluid, skin, oral swabs or any other biological material reflective of the dd-cfDNA.

Dd-cfDNA may be measured by any means known in the art for measurement of dd-cfDNA. For example, methods known in the art include those described in: T. M. Snyder, K. K. Khush, H. A. Valantine, S. R. Quake, Universal noninvasive detection of solid organ transplant rejection. Proc. Natl. Acad. Sci. U.S.A 108, 6229-6234 (2011); Beck et al., Digital droplet PCR for rapid quantification of donor DNA in the circulation of transplant recipients as a potential universal biomarker of graft injury. Clinical chemistry 59, 1732-1741 (2013); De Vlaminck et al., Circulating cell-free DNA enables noninvasive diagnosis of heart transplant rejection. Sci. Transl. Med. 6, 241ra277 (2014); Sigdel et al., A rapid noninvasive assay for the detection of renal transplant injury. Transplantation 96, 97-101 (2013); and Bloom et al., Cell-Free DNA and Active Rejection in Kidney Allografts. J Am Soc Nephrol, (2017).

In one embodiment, the dd-cfDNA assessment method is a single nucleotide polymorphism (SNP)-based methodology. For example, an SNP-based massively multiplexed polymerase chain reaction assay may be used to detect the dd-cfDNA. Such methodologies may be used to determine the percentage of dd-cfDNA with greater precision than other known methodologies, and most advantageously, do not require prior knowledge of donor and recipient genotypes. These methods are also robust across related and unrelated donors. In these methods, samples are processed with PCR amplification of a selected set of SNPs, for example, thousands of SNPs, for example, over 10,000 SNPs. The PCR amplicons are sequenced and the results are analyzed with a probability model to estimate the percentage of dd-cfDNA in the subject.

Exemplary SNP-based models include methods such as those disclosed in: Dar et al., Clinical experience and follow-up with large scale single-nucleotide polymorphism-based noninvasive prenatal aneuploidy testing. Am. J. Obstet. Gynecol. 211, e1-e17 (2014); A. Ryan et al., Validation of an Enhanced Version of a Single-Nucleotide Polymorphism-Based Noninvasive Prenatal Test for Detection of Fetal Aneuploidies. Fetal Diagn. Ther. 40, 219-223 (2016); Pergament et al., Single-nucleotide polymorphism-based noninvasive prenatal screening in a high-risk and low-risk cohort. Obstet. Gynecol. 124, 210-218 (2014); and Altug et al., Analytical Validation of a Single-nucleotide Polymorphism-based Donor-derived Cell-free DNA Assay for Detecting Rejection in Kidney Transplant Patients, Transplantation 1031 2657-2665.

Typically, dd-cfDNA values are expressed as a percentage, wherein the value refers to the percentage of donor-derived DNA of the total cell-free DNA in the sample.

Methods of Assessing Active Rejection. In a first aspect, the scope of the invention encompasses a novel method of identifying active rejection status in a graft recipient by means of dd-cfDNA, wherein the active rejection status encompasses ABMR, TCMR, and ABMR and/or TCMR processes. Previously published methods of identifying active rejection in transplant recipients by means of dd-cfDNA were unable to resolve TCMR subjects from stable subjects, teaching an inability of dd-cfDNA detect TCMR. However, the inventors of the present disclosure have determined that the occurrence of active rejection comprising TCMR may be assessed by dd-cfDNA, as disclosed herein.

In another aspect, the scope of the invention encompasses method of detecting the occurrence of active rejection in a subject by dd-cfDNA wherein the threshold values of dd-cfDNA for the determination of active rejection may be substantially higher than in prior diagnostic methods. In previously reported methods, dd-cfDNA thresholds of 1% (i.e., dd-cfDNA of 1% or greater), sometimes as high as 1.2%, have been used as an indicator of active rejection processes for various graft types, including kidney. However, by the novel dd-cfDNA analysis methods and sample pools utilized by the inventors of the present disclosure, alternative thresholds for determination of active rejection are provided. The inventors of the present disclosure have determined that dd-cfDNA is higher in subjects undergoing active graft rejection than previously understood in the art. For example, as disclosed herein, the mean dd-cfDNA was discovered to be greater than 2% for all forms of active rejection, including: ABMR (2.2%), TCMR (2.7%), and mixed ABMR/TCMR subjects (2.6%). Accordingly, in one embodiment, the invention encompasses a method of identifying subjects with active rejection comprising ABMR, TCMR, or mixed ABMR/TCMR processes by measurements of dd-cfDNA greater than previously established thresholds, e.g. 1%.

Advantageously, the higher thresholds identified herein provide greater sensitivity and specificity in the detection of active rejection. This is especially valuable for the prevention of overtreatment. By the novel disclosures herein, higher thresholds are identified that reduce the risk of unnecessary interventions, which may be invasive, traumatic, or costly. For example, as demonstrated herein, specificity and sensitivity in the detection of active in kidney subjects, can be optimized at a dd-cfDNA threshold of about 1.5% (e.g., in the range of 1.4-1.6).

In one implementation, the scope of the invention encompasses a method of detecting the occurrence of active rejection process in a graft recipient, the methods comprising the steps of
obtaining a sample from a graft recipient;
measuring the abundance of dd-cfDNA in the sample; and
determining if an active rejection process is occurring in the graft recipient, wherein if the measured dd-cfDNA abundance meets or exceeds a selected AR threshold value, active rejection process is determined to be occurring in the graft recipient and if the value of the measured dd-cfDNA is less than the selected threshold value, it is indicative that the graft recipient is not experiencing ongoing active rejection processes.

In certain embodiments: the sample is blood; the graft recipient is a human; the subject is a kidney recipient; and/or the active rejection process is any of ABMR, TCMR, and/or mixed ABMR-TCMR. In various embodiments, the selected threshold AR value is between 1.0 and 2.5%, for example, a threshold dd-cfDNA value selected from the group consisting of 1.0%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75% 1.8%, 1.85%, 1.9%, 1.95%, 2.0%, 2.05%, 2.1%, 2.15%, 2.2%, 2.25%, 2.3%, 2.35%, 2.4%, 2.45% or 2.5%.

In certain embodiments, the scope of the invention encompasses the performance of additional diagnostic and/or treatment steps following assessment of graft rejection status by dd-cfDNA. In one implementation, if the subject is determined to have the occurrence of active rejection, the method comprises the additional step of performing diagnostic tests to confirm the diagnosis of active rejection and/or determine the form or subtype of active rejection. For example, the additional step may be the performance of one or more diagnostic tests to determine if the detected active rejection is ABMR, TCMR, or mixed ABMR/TCMR. In one embodiment, the one or more diagnostic tests is performed by obtaining an additional sample from the graft recipient. In one embodiment, the one or more diagnostic tests is performed on the previously obtained sample utilized to determine active rejection type. In one embodiment, the one or more diagnostic tests comprises a histological analysis performed on a biopsy.

Exemplary histological indicators of ABMR include, for example, microvascular inflammation; peritubular/glomerular basement membrane changes; positive antibody staining for C4d, and the presence of glomerulonephritis, the presence of circulating donor specific antibodies. Exemplary histological indicators of TCMR include, for example, the presence of tubulo-interstitial immune infiltrates; microvascular inflammation; peritubular capillaritis, and glomerulitis. In one embodiment, the one or more diagnostic tests comprises a molecular analysis, for example, a test which assesses the presence of a biomarkers for ABMR or TCMR, for example, a metabolite or the expression of indicator genes. In one embodiment, the one or more additional tests measures the expression of genes indicative of AR or AR subtypes, for example, as described in Sigdel et al., Assessment of 19 Genes and Validation of CRM Gene Panel for Quantitative Transcriptional Analysis of Molecular Rejection and Inflammation in Archival Kidney Transplant Biopsies, Front. Med., 1 Oct. 2019, https://doi.org/10.3389/fined.2019.00213. For example, a significant increase in the expression of INPP5D, ISG20, NKG7, RUNX3, CD31, CD4, CD68, and COL4A is associated with ABMR. A significant increase in the expression of BASP1, CXCL10, CXCL9, INPP5D, ISG20, LCK, RUNX3, CD6, CD4, COL4A is associated with TCMR.

In some implementations, the scope of the invention encompasses a method of treating active rejection in a subject. The method of treating active rejection encompasses the detection of active rejection, as described herein, followed by performance of one or more diagnostic tests to determine which form of active rejection is occurring. Upon determination of the form of active rejection that is occurring, one or more appropriate treatments may be selected and administered to the transplant recipient. For example, In one embodiment, if the risk of TCMR is found to be elevated, treatments appropriate for mitigating TCMR are administered, such as the use of corticosteroids and T cell-depleting agents. In one embodiment, if the risk of ABMR is elevated, treatments appropriate for treating ABMR are applied, for example, plasmapheresis, administration of intravenous immune globulin, and B cell depletion.

Detection of Subacute Rejection. The scope of the invention further encompasses methods of detecting the occurrence of subacute rejection. The methods is based on the novel discovery that the occurrence of Subacute Rejection processes may be determined by assessment of dd-cfDNA. Measured dd-cfDNA values falling within certain ranges will be indicative of ongoing subacute rejection processes. Overall, the occurrence of subacute rejection processes is shown herein to be associated with dd-cfDNA levels intermediate between those observed in stable subjects and those observed in subjects undergoing active rejection. The discovery of an intermediate dd-cfDNA range associated with subacute rejection provides the art with a novel means of diagnosing this category of graft injuries.

In one implementation, the scope of the invention encompasses a method of detecting the occurrence of a subacute rejection process in a graft recipient, the method comprising the steps of
obtaining a sample from a graft recipient;
measuring the abundance of dd-cfDNA in the sample; and
determining if a subacute rejection process is occurring in the graft recipient, wherein if the measured dd-cfDNA abundance falls within a selected subacute rejection range, subacute rejection is determined to be occurring.

In certain embodiments: the sample is blood; the graft recipient is a human; and the graft recipient is a kidney recipient. In one implementation, the graft type is kidney and the subacute rejection may comprise Borderline Rejection. In one embodiment, the subacute rejection is an “Other Rejection” (OR), non-borderline impairment or injury of the graft short of or different from active rejection processes. OR may encompass immune-mediated injury, infection, or drug toxicity effects. In one embodiment, OR includes Early acute rejection without any clinical graft dysfunction-also called subclinical AR, which is often only picked up by protocol biopsy. In one embodiment, OR encompasses viral infection, for example, infection by a polyomavirus family member, for example, a BK virus or BKV.

The selected dd-cfDNA range indicative of subacute rejection will comprise a minimum value and a maximum value, wherein, if the measured dd-cfDNA value falls within the range, it is equal to or greater than the minimum value and is equal to or less than the maximum value. In various embodiments, the minimum value is between 0.25 and 0.8%, including all intermediate values within this range, for example, a minimum value selected from the group consisting of 0.25%, 0.3%, 0.4%, 0.5%, 0.6%, 0.7%, or 0.8%. In one embodiment, the maximum value of the range is between 0.75% and 1.75%, including all intermediate values within this range, for example, a maximum value selected from the group consisting of 0.75%, 0.8%, 0.85%, 0.9%, 0.95%, 1.0%, 1.05%, 1.1%, 1.15%, 1.2%, 1.25%, 1.3%, 1.35%, 1.4%, 1.45%, 1.5%, 1.55%, 1.6%, 1.65%, 1.7%, and 1.75%. In one embodiment, the minimum value of the selected dd-cfDNA range indicative of subacute rejection is 0.25% and the maximum value of the range is 1.75%. In one embodiment, the minimum value of the selected dd-cfDNA range indicative of subacute rejection is 0.5% and the maximum value of the range is 1.5%. In one embodiment, the minimum value of the selected dd-cfDNA range indicative of subacute rejection is 0.5% and the maximum value of the range is 1.0%. In one embodiment, the minimum value of the selected dd-cfDNA range indicative of subacute rejection is 0.5% and the maximum value of the range is 1.25%. In one embodiment, the minimum value of the selected dd-cfDNA range indicative of subacute rejection is 0.5% and the maximum value of the range is 0.5 and the maximum value of the range is 1.5%.

In certain embodiments, the method encompasses the performance of additional diagnostic and/or interventions. In one implementation, if the subject is determined to have the occurrence of subacute rejection, the method comprises the additional step of performing diagnostic tests to confirm the diagnosis of subacute rejection and/or determine the form or subtype of subacute rejection. For example, in one embodiment, the graft recipient is a kidney recipient and the additional step is performance of one or more diagnostic tests to determine if the detected active rejection is borderline rejection or OI. In one embodiment, the one or more diagnostic tests is performed by obtaining an additional sample from the graft recipient. In one embodiment, the one or more diagnostic tests is performed on the previously obtained sample utilized to determine subacute rejection type. In one embodiment, the one or more diagnostic tests comprises a histological analysis performed on a biopsy, for example a kidney biopsy.

When measured dd-cfDNA indicates subacute rejection by the methods of the invention, various additional steps may be performed. In one embodiment, additional assays may be performed to verify subacute rejection. For example, a biopsy may be performed, wherein the results of the biopsy can confirm the subacute rejection diagnosis. For example, viral infection may be determined in tissue samples by viral inclusions on biopsy, positive staining for SV40 antigen, or circulating DNAemia. Other diagnostic tools may be applied to determine OI occurrence and type, such as detecting viral infection by blood PCR analysis or detecting drug toxicity by measurement of high drug levels in blood.

In one aspect, the scope of the invention encompasses methods of treating subacute rejection in a transplant recipient. The method of treatment comprises the steps of:
determining, by the dd-cfDNA analytical methods disclosed herein, that subacute rejection is occurring in a transplant recipient; and
administering one or more suitable treatments or intervention to treat the detected subacute rejection.
   The treatment or intervention may comprise any process that ameliorates the symptoms of the subacute rejection or which otherwise improves graft function and survival. In one embodiment, the intervention is performance of additional monitoring of dd-cfDNA, graft function, or graft injury, for example, at weekly, monthly or other selected intervals. In the case of kidney transplant, if borderline rejection occurrence is assessed, the graft recipient may be treated with additional steroids or augmentation of maintenance immunosuppression, or by other borderline rejection therapeutic treatments known in the art. For example, in one embodiment borderline rejection is treated by increasing immunosuppression dosing or by giving a course, e.g. a 3 days course, of higher dose steroid. It has been shown that untreated subclinical rejection, e.g., Banff graded rejection but without a change in graft function- or borderline rejection, e.g. rejection with or without graft dysfunction but not meeting all the Banff classification to define full blown rejection—can develop into full clinical rejection and increase risk of developing chronic rejection or increased risk of premature graft loss. Accordingly, additional monitoring and/or treatment may be administered to prevent such outcomes in subjects found to have the occurrence of subacute rejection.

Combined Measures for Assessment of Kidney Graft Status. In one implementation, the dd-cfDNA measurements of the invention are combined with a measure of kidney function, such as creatinine or estimated glomerular filtration rate (eGFR) score to discriminate between stable status, subacute status, and active rejection status. In measurements of dd-cfDNA, subacute and stable dd-cfDNA values tend to segregate together. In measurements of kidney function (e.g. eGFR), subacute rejection values tend to stratify with active rejection values. Accordingly, in one implementation, the scope of the invention encompasses a method of determining if a transplant recipient is stable, undergoing subacute rejection, or undergoing active rejection by the following steps:
measuring dd-cfDNA in the graft recipient by assessment of a sample obtained the kidney recipient;
obtaining a measurement of kidney function by assessment of a sample obtained the kidney recipient;
and determining kidney transplant status; wherein if the measured dd-cfDNA value exceeds a selected dd-cfDNA active rejection threshold value, the subject is deemed to be undergoing active rejection;
if the measure dd-cfDNA value is less than the selected dd-cfDNA active rejection threshold value; and if the measure of kidney function is indicative of normal kidney function, the subject is deemed to be stable; and if the measured dd-cfDNA value is less than the selected dd-cfDNA active rejection threshold value; and if the measure of kidney function is indicative of impaired kidney function, the subject is deemed to be undergoing borderline rejection.

In one embodiment, the dd-cfDNA threshold active rejection value is a value between 0.75 and 2, for example, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95 and 2.0.

In one embodiment, the measure of kidney function is eGFR score. eGFR may be calculated as known in the art, for example, from creatinine measured serum creatinine values, and other factors such as lean body mass, age, race, gender, weight, and other factors used to calculate eGFR as known in the art. In various embodiments, the selected threshold indicative of normal kidney function is an eGFR value between 60 and 100, for example, 60, 65, 70, 75, 80, 85, 90, 95, or 100, wherein a calculated eGFR score above the selected threshold is indicative of normal kidney function, and a calculated eGFR score below the selected threshold is indicative of impaired kidney function. Conveniently, in one embodiment, the eGFR score and dd-cfDNA measurements may be obtained from a single blood sample.

In one embodiment, the measure of kidney function is creatinine serum level. In various embodiments, the selected threshold indicative of normal kidney function is a creatinine serum value between 0.4 and 1.3 mg/dl, for example, a value selected from the group consisting of 0.4, 0.5, 0.6, 0.7, 0.8, 0.9, 1.0, 1.1, 1.2, or 1.3 mg/dl, wherein a measured serum creatinine concentration above the selected threshold is indicative of impaired kidney function and a measured serum creatinine concentration below the selected threshold is indicative of normal kidney function.

In some implementations, the scope of the invention encompasses a method of treating subacute rejection or active rejection in a kidney recipient by the steps of: performing the combined measurement method of the invention to identify kidney recipient as stable, undergoing active rejection, or undergoing subacute rejection, wherein, if the kidney recipient is determined to have subacute rejection, a suitable intervention for subacute rejection is administered and if the subject is determined to have active rejection, a suitable intervention for active rejection is administered. In the case of subacute rejection, the suitable intervention may encompass diagnostic measures to differentiate between borderline rejection, viral infection, or other kidney injury, followed by administration of an appropriate intervention based on the type of subacute rejection found. In the case of active rejection, the suitable intervention may encompass diagnostic measures to differentiate between ABMR, TCMR, or combined ABMR/TCMR, followed by administration of an appropriate intervention based on the type of active rejection found.

Application of the Methods. The methods of the invention may be applied to recipients under various regimens. In one embodiment, dd-cfDNA is measured and graft rejection status is assessed at regular intervals, e.g. weekly, monthly, annually, etc., following transplant to monitor for potential subacute and/or active rejection. In one embodiment, the dd-cfDNA measurements and rejection status assessments are performed upon presentation of symptoms consistent with a potential subacute or acute rejection process. In some implementations, the dd-cfDNA measurement and rejection status assessment methods of the invention are combined with one or more additional diagnostic tools known in the art in order to improve the resolution of the assessments.

The methods of the invention are effective for subject having a first transplant or multiple transplants. For example, in the case of renal transplants, the methods may be applied to subjects having a first graft or those with multiple or serial renal transplants.

### Exemplary Embodiments

In various embodiments, the scope of the invention encompasses a method of detecting the occurrence of active rejection process in a graft recipient, the methods comprising the steps of: obtaining a sample from a graft recipient; measuring the abundance of dd-cfDNA in the graft recipient by the sample; and determining if an active rejection process is occurring in the graft recipient, wherein if the measured dd-cfDNA abundance meets or exceeds a selected AR threshold value, active rejection process is determined to be occurring in the graft recipient and if the value of the measured dd-cfDNA is less than the selected threshold value, it is indicative that the graft recipient is not experiencing ongoing active rejection processes; wherein the active rejection process comprises antibody mediated rejection, T-Cell mediated rejection, or combined antibody mediated rejection and T-Cell mediated rejection; wherein the graft is a graft selected from the group consisting of kidney, heart, lung, liver, skin, cornea, intestine, pancreas, limb, digit, bone, ligament, cartilage, and tendon; in one embodiment: the graft being a kidney; the sample being a blood sample; the selected threshold active rejection threshold value is between 1.0 and 2.5%; such as a threshold value is between 1.4 and 1.6%; the measurement of dd-cfDNA is achieved by a SNP analysis; in one embodiment the SNP analysis encompasses the use of at least 10,000 SNPs; in some embodiments, the genotype information of the donor is unknown and/or the donor and recipient are related.

In various embodiments, the scope of the invention encompasses a method of treating active rejection in a graft recipient, comprising the steps of detecting the occurrence of active rejection process in the graft recipient by the methods disclosed herein and administering a suitable intervention to the graft recipient if active rejection is detected.

In various embodiments, the scope of the invention encompasses method of detecting the occurrence of a subacute rejection the methods comprising the steps of: obtaining a sample from a graft recipient; measuring the abundance of dd-cfDNA in the graft recipient by the sample; and

determining if a subacute rejection process is occurring in the graft recipient, wherein if the measured dd-cfDNA abundance falls within a selected subacute rejection range intermediate between dd-cfDNA values measured in stable subjects and dd-cfDNA values measured in subjects undergoing active rejection, subacute rejection is determined to be occurring; wherein the graft is a graft selected from the group consisting of kidney, heart, lung, liver, skin, cornea, intestine, pancreas, limb, digit, bone, ligament, cartilage, and tendon; being a kidney in one embodiment; in one embodiment, the sample being a blood sample; wherein, in various embodiments, the selected dd-cfDNA range indicative of subacute rejection is between 0.5 and 1.5%; in various embodiments the minimum value of the range is between 0.25 and 0.8%, including all intermediate values within this range, for example, a minimum value selected from the group consisting of 0.25%, 0.3%, 0.4%, 0.5%, 0.6%, 0.7%, or 0.8%; in various embodiments, the maximum value of the range is between 0.75% and 1.75%, including all intermediate values within this range, for example, a maximum value selected from the group consisting of 0.75%, 0.8%, 0.85%, 0.9%, 0.95%, 1.0%, 1.05%, 1.1%, 1.15%, 1.2%, 1.25%, 1.3%, 1.35%, 1.4%, 1.45%, 1.5%, 1.55%, 1.6%, 1.65%, 1.7%, and 1.75%; wherein in some embodiments, the measurement of dd-cfDNA is achieved by a SNP analysis; in one embodiment, the SNP analysis encompasses the use of at least 10,000 SNPs; in one embodiment, the genotype information of the donor is unknown; and in one embodiment the donor and recipient are related.

In various embodiments, the scope of the invention encompasses methods of assessing graft rejection status in a kidney recipient, comprising the steps of: measuring dd-cfDNA in the graft recipient by assessment of a sample obtained the kidney recipient; obtaining a measurement of kidney function by assessment of a sample obtained the kidney recipient; and determining kidney transplant status; wherein if the measured dd-cfDNA value exceeds a selected dd-cfDNA active rejection threshold value, the subject is deemed to be undergoing active rejection; if the measure dd-cfDNA value is less than the selected dd-cfDNA active rejection threshold value; and if the measure of kidney function is indicative of normal kidney function, the subject is deemed to be stable; and if the measured dd-cfDNA value is less than the selected dd-cfDNA active rejection threshold value; and if the measure of kidney function is indicative of impaired kidney function, the subject is deemed to be undergoing borderline rejection; in one embodiment, the dd-cfDNA threshold active rejection value is a value between 0.75% and 2%, in one embodiment, the dd-cfDNA threshold active rejection value being selected from 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95 and 2.0, in one embodiment, the dd-cfDNA threshold active rejection value is 1%; in one embodiment, the dd-cfDNA threshold active rejection value is 1.5%; in various embodiments the measure of kidney function is eGFR score 60 or greater is indicative of normal kidney function; in various embodiments a creatinine serum value below 1.3 is indicative of normal kidney function.

### EXAMPLES

### Example 1. Optimizing Detection of Kidney Transplant Injury by Assessment of Donor-Derived Cell-Free DNA Via Massively Multiplex PCR

Study Population and Samples. Male and female adult or young adult patients received a kidney from related or unrelated living donors, or unrelated deceased donors. Plasma samples were obtained from an existing biorepository; time points of patient blood draw following transplantation surgery were either at the time of an allograft biopsy or at various pre-specified time intervals based on lab protocols. Typically, samples were biopsy-matched at time of clinical dysfunction and biopsy or at the time of protocol biopsy, at which time most patients did not have clinical dysfunction. In addition, some patients had serial post transplantation blood drawn. The selection of study samples was based on (a) adequate plasma being available, and (b) if the sample was associated with biopsy information. Among study samples, 72.3% were drawn on the day of biopsy. Patients without biopsy-matched samples were excluded from the primary analyses.

Biopsy Samples. All kidney biopsies were analyzed in a blinded manner by a trained pathologist and were graded by the 2017 Banff classification [18] for active rejection (AR); intragraft C4d stains were performed [19] to assess for acute humoral rejection [20]. Biopsies are not done in cases of active UTI or other infections. Transplant “injury” was defined as a >20% increase in serum creatinine from its previous steady-state baseline value and an associated biopsy that was classified as either active rejection (AR), borderline rejection (BL), or other injury (OI) (e.g., drug toxicity, viral infection). AR was defined, at minimum, by the following criteria: (1) T-cell-mediated rejection (TCMR) consisting of either a tubulitis (t) score >2 accompanied by an interstitial inflammation (i) score >2 or vascular changes (v) score >0; (2) C4d positive antibody-mediated rejection (ABMR) consisting of positive donor specific antibodies (DSA) with a glomerulitis (g) score >0/or peritubular capillaritis score (ptc) >0 or v>0 with unexplained acute tubular necrosis/thrombotic micro angiopathy (ATN/TMA) with C4d=2; or (3) C4d negative ABMR consisting of positive DSA with unexplained ATN/TMA with g+ptc >2 and C4d is either 0 or 1. Borderline change (BL) was defined by t1+i0, or t1+i1, or t2+i0 without explained cause (e.g., polyomavirus-associated nephropathy (PVAN)/infectious cause/ATN). Other criteria used for BL changes wereg>0 and/or ptc >0, orv>0 without DSA, or C4d or positive DSA, or positive C4d without nonzero g or ptc scores. Normal (STA) allografts were defined by an absence of significant injury pathology as defined by Banff schema.

2.4. dd-cfDNA Measurement in Blood Samples. Cell-free DNA was extracted from plasma samples using the QUIAAMP™ Circulating Nucleic Acid Kit (Qiagen) and quantified on the LABCHIP™ NGS 5 k kit (Perkin Elmer) following manufacturer's instructions. cfDNA was input into library preparation using the library preparation kit as described in Abbosh et al., Phylogenetic ctDNA analysis depicts early-stage lung cancer evolution. Nature 2017, 545, 446-451, with a modification of 18 cycles of library amplification to plateau the libraries. Purified libraries were quantified using and target enrichment was accomplished using massively multiplexed-PCR (mmPCR) using a modified version of the method described in Zimmermann et al., Noninvasive prenatal aneuploidy testing of chromosomes 13, 18, 21, X, and Y, using targeted sequencing of polymorphic loci. Prenat. Diagn. 2012, 32, 1233-1241, with 13,392 single nucleotide polymorphisms (SNPs) targeted. Amplicons were then sequenced on an Illumina HISEQ™ 2500 Rapid Run, 50 cycles single end, with 10-11 million reads per sample.

Statistical Analyses of dd-cfDNA and eGFR. In each sample, dd-cfDNA was measured and correlated with rejection status, and results were compared with eGFR. Where applicable, all tests were two sided. Significance was set at p<0.05. Because the distribution of dd-cfDNA in patients was severely skewed among the groups, data were analyzed using a Kruskal-Wallis rank sum test followed by Dunn multiple comparison tests with Holm correction eGFR (creatinine in mg/dL) was calculated as known in the art, briefly, eGFR=186×Serum Creatinine⁻¹⁵⁴×Age^{-0.203}×(1.210 if Black)×(0.742 if Female). To evaluate the performance of dd-cfDNA and eGFR (mL/min/1.73 m²) as rejection markers, samples were separated into an AR group and a non-rejection group (BL+STA+OI). Using this categorization, the following predetermined cut-offs were used to classify as sample as AR: >1% for dd-cfDNA and <60.0% for eGFR. To calculate the performance parameters of each marker (sensitivity, specificity, positive predictive value (PPV), negative predictive value (NPV), and area under the curve (AUC)), a bootstrap method was used to account for repeated measurements within a patient. Briefly, at each bootstrap step, a single sample was selected from each patient; by assuming independence among patients, the performance parameters and their standard errors were calculated. This was repeated 10,000 times; final confidence intervals were calculated using the bootstrap mean for the parameter with the average of the bootstrap standard errors with standard normal quantiles. Standard errors for sensitivity and specificity were calculated assuming a binomial distribution; for PPV and NPV a normal approximation was used; and for AUC the DeLong method was used. Performance was calculated for all samples with a matched biopsy, and for the sub-cohort consisting of samples drawn at the same time as a protocol biopsy. Differences in dd-cfDNA levels by donor type (living related, living non-related, and deceased non-related) were also evaluated. Significance was determined using the Kruskal-Wallis rank sum test as described above. Inter- and intra-variability in dd-cfDNA over time was evaluated using a mixed effects model with a logarithmic transformation on dd-cfDNA; 95% confidence intervals for the intra- and inter-patient standard deviations were calculated using a likelihood profile method. Post hoc analyses evaluated (a) different dd-cfDNA thresholds to maximize NPV and (b) combined dd-cfDNA and eGFR to define an empirical rejection zone that may improve the PPV for AR diagnosis.

Results. Patients and Blood Samples. A total of 300 plasma samples were collected from 193 unique renal transplant recipients. Of these, 23 samples from 15 patients did not meet inclusion criteria and were excluded from analyses; this included samples collected within three days from transplant (15), and samples unable to be sequenced (8). Of the remaining 277 samples, 217 were biopsy-matched, including 38 collected from patients with biopsy-proven active rejection (AR), 72 with biopsy-proven borderline rejection (BL), 82 normal, stable allografts (STA), and 25 with a biopsy that indicated other injury (OI). Of the 178 unique patients included in the study, 20% (35) were under 18 years of age; 30% (54) were between 18 and 40 years, and 50% (89) were older than 40 years of age at the time of first blood sample.

Published data have shown that dd-cfDNA fractions in patients with AR are significantly higher than patients with non-rejection; however, these data have shown an inability of dd-cfDNA to detect all types of AR, specifically failing to detect TCMR. In this data set, the performance of the assay to detect rejection was evaluated for all types of rejection combined (ABMR, TCMR), based on the assumption that elevated dd-cfDNA levels are indicative of ongoing damage to the transplanted organ, irrespective of the underlying biology of rejection. Therefore, the ability of the assay to detect AR versus non-rejection was calculated, where non-rejection was defined as all specimens that were classified as STA, BL, or OI. Additionally, the performance of the assay to discriminate AR from complete absence of injury (STA) was also evaluated. A summary of demographic information and sample characteristics are provided in Table 1. All pathology samples were read at UCSF by a single renal pathologist and rated according to the recently updated Banff criteria, as described in Haas, et al. The Banff 2017 Kidney Meeting Report: Revised diagnostic criteria for chronic active T cell-mediated rejection, antibody-mediated rejection, and prospects for integrative endpoints for next-generation clinical trials. Am. J. Transplant. 2018, 18, 293-307.

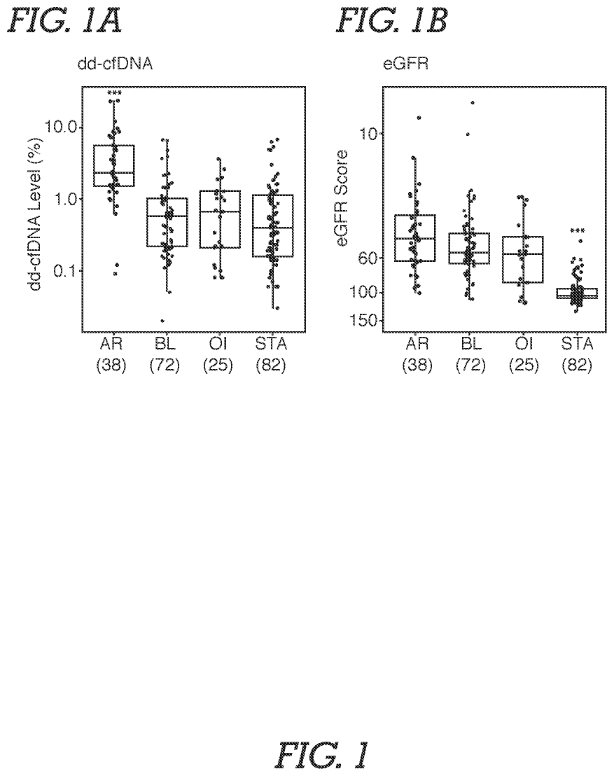

dd-cfDNA and eGFR in Kidney Transplant Recipients. The amount of dd-cfDNA was significantly higher in the circulating plasma of the AR group (median=2.32%) compared with the non-rejection group (median=0.47%, p<0.0001) (Table 2). Additionally, the median level of dd-cfDNA was significantly higher in the AR group compared with all three individual non-rejection subgroups: BL group (0.58%), STA group (0.40%), and OI (0.67%, all comparisons, adj. p<0.0001) (FIG. 1A). That the dd-cfDNA burden was higher in the AR group as compared to the BL group indicates that dd-cfDNA fraction may be used to track the evolution of early injury to more established rejection, as well as any subsequent recovery.

In contrast to dd-cfDNA, eGFR scores did not have as much discriminatory ability for differentiating AR and individual non-rejection groups (Table 2). Overall, the median eGFR score in the AR group (45.67) was significantly lower than that observed in the non-rejection group (76.6, p<0.0001) (Table 2) and even lower compared to the STA group alone (104.5, adj. p<0.0001) (Table 2, FIG. 1B). However, unlike the dd-cfDNA results, there was no difference in median eGFR scores between the AR and BL groups (45.67 vs. 55.99, adj. p=0.461) (Table 2; FIG. 1B). Additionally, compared with the STA group, eGFR levels were significantly higher in the BL (55.99, adj. p<0.0001) and OI (57.4, adj. p<0.0001) groups (Table 2, FIG. 1B).

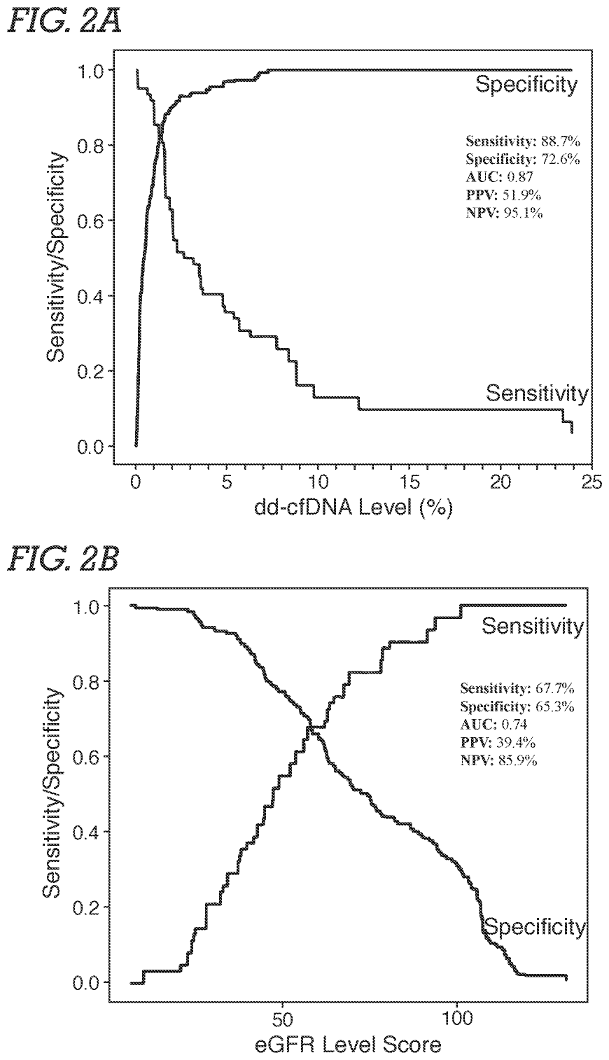

Performance Estimates for Discriminatory Ability of Tests. With a dd-cfDNA cutoff of >1%, the mmPCR-NGS method had an 88.7% sensitivity (95% confidence interval (CI), 77.7-99.8%) and 72.6% specificity (95% CI, 65.4-79.8%) for detection of AR. Sensitivity and specificity values are shown over the range of dd-cfDNA cutoffs in FIG. 2A. The area under the curve (AUC) was 0.87 (95% CI, 0.80-0.95). Based on a 25% prevalence of rejection in an at-risk population, the positive predictive value (PPV) was projected to be 52.0% (95% CI, 44.7-59.2%) and the negative predictive value (NPV) was projected to be 95.1% (95% CI, 90.5-99.7%).

Sensitivity and specificity were lower using eGFR (FIG. 2B). Using an eGFR cutoff score <60 for AR, sensitivity and specificity values were 67.8% (95% CI, 51.3-84.2%) and 65.3% (57.6-73.0%), respectively, with an AUC of 0.74 (0.66-0.83). The projected PPV and NPV values of eGFR were 39.4% (31.6-47.3%) and 85.9% (75.9-92.2%), respectively.

As a post hoc analysis, the combination of eGFR with dd-cfDNA was evaluated. Samples with a very high eGFR score, for example, tended to correspond to non-rejection samples. Defining the active rejection zone to be dd-cfDNA level >1% and eGFR <100, and non-rejection to be dd-cfDNA level <1% or eGFR >100, the combined dd-cfDNA and eGFR markers correctly classified 32/38 (84.2%) AR samples, and 145/179 (81.0%) non-rejection samples. Meanwhile at an equivalent specificity of 81.0%, (using a cut off of 1.3% dd-cfDNA) the sensitivity of the dd-cfDNA marker alone was 82.3%. Therefore the combined biomarker approach appeared to add little or no value over cfDNA alone.

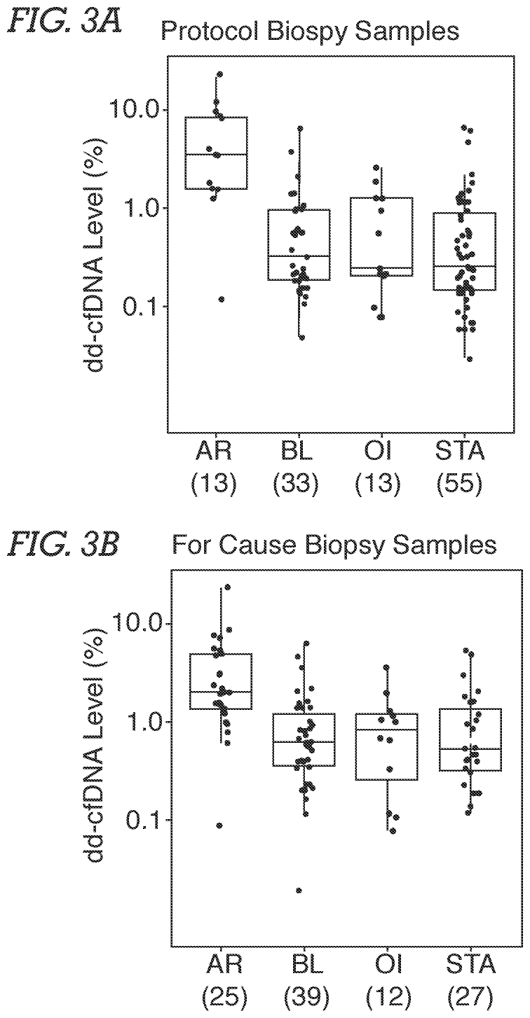

dd-cfDNA Performance in Unique Biopsy-Confirmed Subgroups. Among the biopsy-matched samples, 103 (47.5%) were biopsied for clinical reasons, whereas 114 (52.5%) were biopsied according to protocol. FIG. 3 depicts sample dd-cfDNA levels among all subgroups; 85 (39.2%) had dd-cfDNA levels >1%. Of those, 22 (25.9%) were STA; the remainder were AR (33 (38.8%)), OI (10 (11.8%)), or BL (20 (23.5%)). Of the individual groups, 33 (86.8%) of the total AR samples and 22 (26.8%) of the total STA samples had dd-cfDNA levels above 1%. In comparison, 20 (27.8%) of the total BL samples and 10 (40.0%) of the total OI samples had dd-cfDNA levels above 1%.

FIGS. 3A and 3B show assay performance for the subset of samples drawn at the time of a for-cause biopsy (FIG. 3A) and protocol biopsy (FIG. 3B); performance shown in protocol biopsies is expected to reflect performance when the assay is used in routine surveillance, that is, when there are no signs of renal injury. This cohort of 114 samples showed a 92.3% sensitivity (95% CI, 64.0-99.8%) and 75.2% specificity (95% CI, 65.7-83.3%) for detection of AR. The area under the curve (AUC) was 0.89 (95% CI, 0.76-0.99). Based on a 25% prevalence of rejection in an at-risk population, the positive predictive value (PPV) was projected to be 55.4% (95% CI, 46.2-64.7%) and the negative predictive value (NPV) was projected to be 96.7% (95% CI, 90.6-99.9%).

Sensitivity, specificity, PPV and NPV were also calculated at different dd-cfDNA level rejection cutoffs, e.g. at 0.6%, 0.8%, 1.0%, 1.2%, 1.4% and 1.6%. Raising the cutoff has the effect of improving the specificity and the PPV; lowering the cutoff improved sensitivity and NPV.

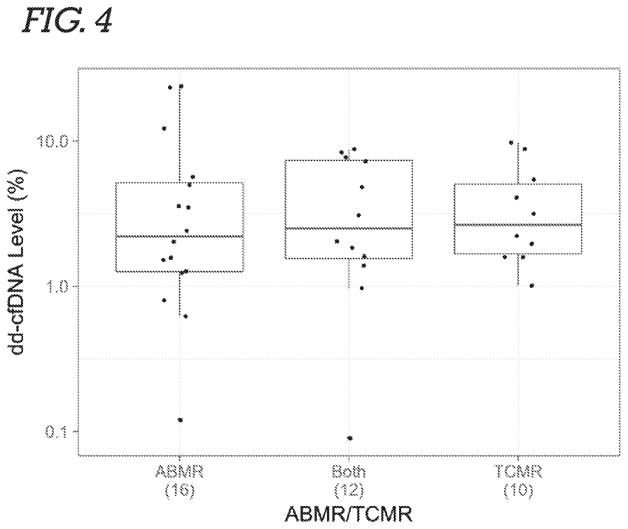

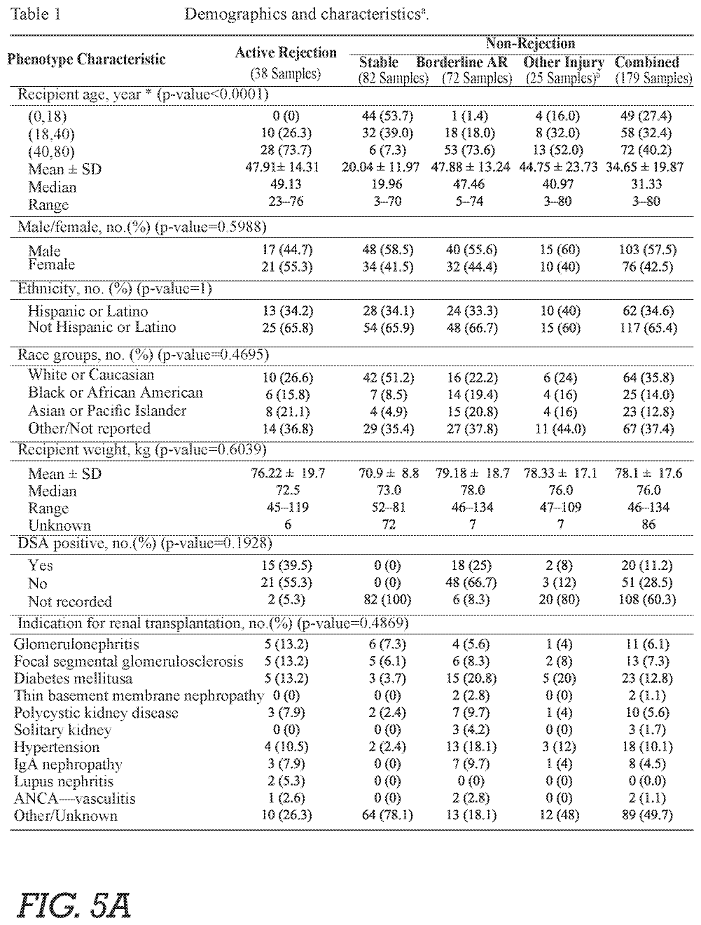

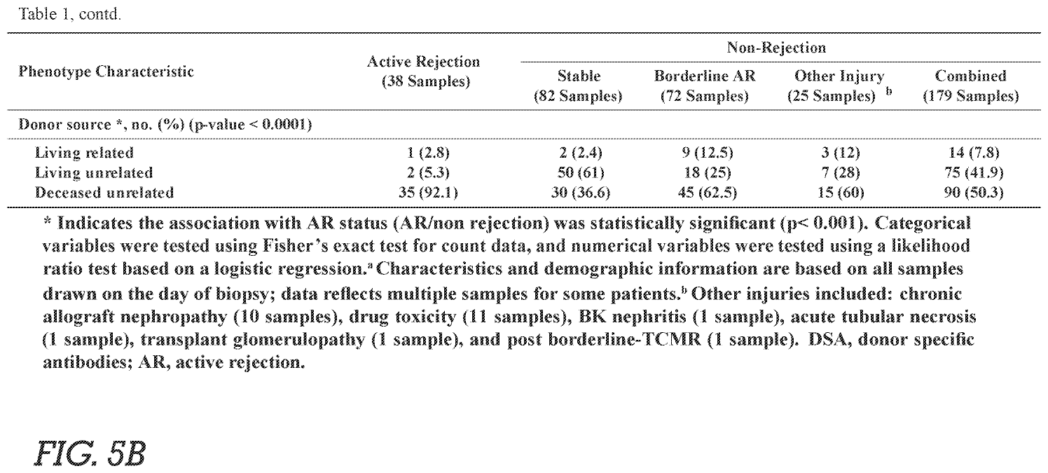

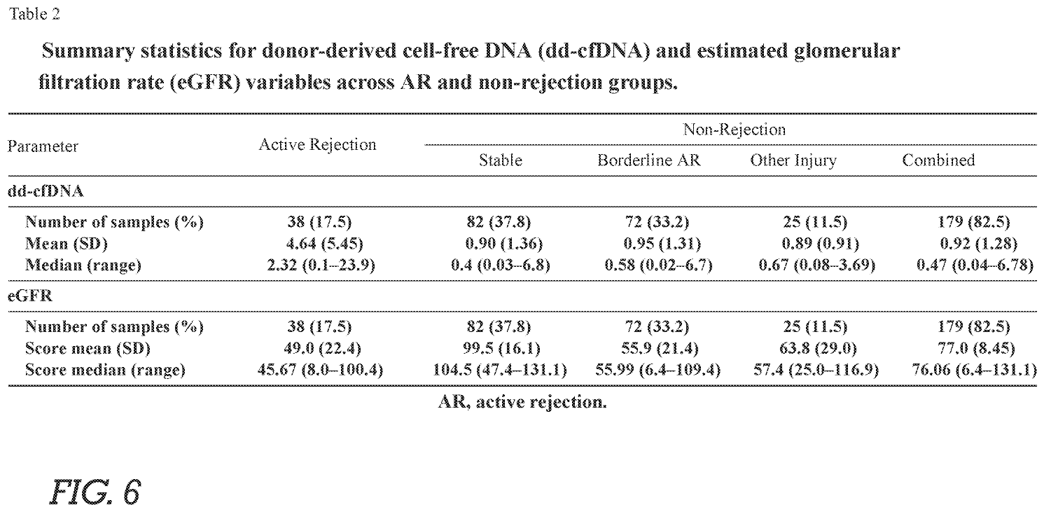

Relationship Between dd-cfDNA and Rejection Type. Of the 38 samples with biopsy-proven AR, 16 were classified as either antibody-mediated rejection (ABMR) or ABMR and borderline T-cell-mediated rejection (bTCMR); 12 had a combination of both ABMR and TCMR; 10 were classified as either TCMR or TCMR and bABMR. In addition, 13 and 59 BL samples were classified as bAMBR and bTCMR, respectively. FIG. 4 shows the relationship between dd-cfDNA level and type of rejection. Median dd-cfDNA did not differ significantly between AMBR (2.2%), ABMR/TCMR (2.6%), or TCMR (2.7%) groups (p=0.855) (Table S4). The study contained a range of pathologies, and the data indicate that this assay, unlike other published studies measuring cfDNA by other assays, is robust to different rejection types.

dd-cfDNA Levels by Donor Type. To assess the relationship between dd-cfDNA and donor type (living related, living non-related, and deceased non-related) a linear mixed-effects model was constructed using a log transformed dd-cfDNA as the response and donor type as the predictor for the non-rejection group. The log-transformation was applied to satisfy the model's assumptions. The test was limited to the non-rejection group due to the limited number of AR samples in two groups (living related and living non-related). An ANOVA Wald-test with Kenward-Roger approximation for the degrees of freedom showed significance (p=0.045). Tukey's post-hoc test was used to determine the difference among the three groups: none of the post-hoc tests demonstrated any association.

dd-cfDNA Variability over Time. Two analyses were designed to evaluate the natural variability in dd-cfDNA over time in biopsy-matched, non-rejection patients. The first sub-analysis was a cross-sectional analysis of 60 plasma samples from 60 different patients, collected immediately following surgery (within three days (“0 months”)) or at 1, 3, 6, or 12 months post-surgery. Among these STA patients, dd-cfDNA levels were lower at month 0 than subsequent time points; however, for most of these STA samples dd-cfDNA levels were <1% across all time points. No association was observed between Day 0 samples and the other time points, although the overall distribution of dd-cfDNA levels in the Day 0 group appears lower in comparison. Overall, organ injury occurred at dd-cfDNA levels above 1%. The inter-patient standard deviation within this cohort was 0.16 (95% CI, 0.0-0.37) and the intra-patient standard deviation was 0.42 (95% CI, 0.32-0.56). The intraclass-correlation coefficient was low (0.1193), which suggests that the variability in these data are mostly due to intra-patient variation.

Discussion. In this study, median dd-cfDNA was significantly higher in the AR group (2.32%) versus the non-rejection group (0.47%; p<0.0001). Analysis of performance estimates demonstrated that the mmPCR-NGS method was able to discriminate active from non-rejection status with an AUC of 0.87 and high sensitivity (88.7%) and specificity (72.6%) at the AR cutoff of >1% dd-cfDNA. Based on a 25% prevalence of rejection, projected PPV and NPV were 52.0% and 95.1%, respectively. In contrast, eGFR scores were generally less discriminatory, with a 67.7% sensitivity and 65.3% specificity, and projected PPV and NPV of 39.4% and 85.9%, respectively. Therefore, if eGFR measurements were used as the sole clinical decision point, about 1 in 7 patients found to be at low risk of rejection would actually be experiencing rejection, and would not be referred for an indication biopsy—this is in comparison to the projected NPV for dd-cfDNA that suggests that only 1 in 20 patients would miss an indication biopsy where it might be clinically necessary. Taken together, the superior performance of this SNP-based dd-cfDNA assay over that of the current standard of care for the evaluation of allograft rejection enables patients a greater opportunity for timely therapy in the case of an allograft injury.

levels of dd-cfDNA also provided discrimination of AR from the three non-rejection subgroups (STA, BL, and OI); median dd-cfDNA levels were significantly higher for samples with biopsy-proven AR (2.3%) versus BL (0.6%), OI (0.7%), and STA (0.4%). In a post hoc analysis, the ability of dd-cfDNA combined with eGFR to predict rejection status (AR/non-rejection) in biopsy matched samples was examine. This combined approach correctly classified 32/38 (84.2%) AR and 145/179 (81.0%) non-rejection samples, though in a head-to-head comparison it showed little to no improvement over dd-cfDNA alone. Also of note, while both dd-cfDNA and eGFR can be used to differentiate AR and STA cases, the BL and OI samples stratify differently: they tend to aggregate with STA when using dd-cfDNA and with AR when using eGFR. This demonstrates that dd-cfDNA can be interpreted together with eGFR to differentiate patients into three groups-STA patients, AR patients, and patients experiencing BL or OI.

Another important finding of this study was that the fraction of dd-cfDNA did not differ between ABMR and TCMR groups, with dd-cfDNA levels of 2.2% and 2.7%, respectively. These results are novel considering that the previously conducted study by Bloom et al. (2017), which used a different assay, found significantly higher dd-cfDNA levels for ABMR (2.9%) than for TCMR (51.2%), showing a lower ability to detect T-cell mediated rejections. Though the assay used in that study also measured dd-cfDNA, the methods used by the two assays differ greatly. The dd-cfDNA measurements based on the mmPCR assay in the results disclosed herein can accurately discriminate AR from non-rejection across a range of pathologies, including both acute and chronic findings, in both the ABMR and TCMR groups. An additional finding in this study is that borderline, or early rejection injury, has a lower burden of dd-cfDNA than more established injury, making it possible to use the methods of the invention to track evolution of, or recovery from, active rejection.

One barrier to widespread clinical use of dd-cfDNA as a diagnostic tool for monitoring organ transplant has been the limitations in measuring dd-cfDNA in certain cases, such as when the donor genotype is unknown or when the donor is a close relative. In the methods disclosed herein, advantageously, it is possible to quantify dd-cfDNA without prior recipient or donor genotyping. Further, there is no need for a computational adjustment based on whether the donor is related to the recipient. In the methods disclosed herein, evaluation of dd-cfDNA levels by donor type revealed that regardless of donor type (living related, living non-related, deceased non-related), dd-cfDNA levels were similar across all donor types within in the AR and non-rejection categories.

In conclusion, the results disclosed herein demonstrate the use of dd-cfDNA in the blood as an accurate marker of kidney injury/rejection across a range of pathologies with acute and chronic findings. This rapid, accurate, and noninvasive technology allows for detection of significant renal injury in patients better than the current standard of care, with the potential for better patient management, more targeted biopsies, and improved renal allograft function and survival.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

1. A method of detecting the occurrence of active rejection process in a graft recipient, the methods comprising the steps of
obtaining a sample from a graft recipient;
measuring the abundance of dd-cfDNA in the graft recipient by the sample; and
determining if an active rejection process is occurring in the graft recipient, wherein if the measured dd-cfDNA abundance meets or exceeds a selected AR threshold value, active rejection process is determined to be occurring in the graft recipient and if the value of the measured dd-cfDNA is less than the selected threshold value, it is indicative that the graft recipient is not experiencing ongoing active rejection processes;
wherein the active rejection process comprises antibody mediated rejection, T-Cell mediated rejection, or combined antibody mediated rejection and T-Cell mediated rejection.

2. The method of claim 1, wherein
the graft is a graft selected from the group consisting of kidney, heart, lung, liver, skin, cornea, intestine, pancreas, limb, digit, bone, ligament, cartilage, and tendon.

3. The method of claim 2, wherein
the graft is kidney.

4. The method of claim 1, wherein
the sample is a blood sample.

5. The method of claim 1, wherein
the selected threshold active rejection threshold value is between 1.0 and 2.5%.

6. The method of claim 5, wherein
the selected threshold active rejection threshold value is between 1.4 and 1.6%.

7. The method of claim 1, wherein
the measurement of dd-cfDNA is achieved by a SNP analysis.

8. The method of claim 7, wherein
the SNP analysis encompasses the use of at least 10,000 SNPs.

9. The method of claim 1, wherein
the genotype information of the donor is unknown.

10. The method of claim 1, wherein
the donor and recipient are related.

11. The method of claim 1,
wherein, if the occurrence of an active rejection process is detected, the method encompasses the additional step of
administering a suitable intervention to the graft recipient.

12. A method of detecting the occurrence of a subacute rejection process in a graft recipient, the methods comprising the steps of
obtaining a sample from a graft recipient;
measuring the abundance of dd-cfDNA in the graft recipient by the sample; and
determining if a subacute rejection process is occurring in the graft recipient, wherein if the measured dd-cfDNA abundance falls within a selected subacute rejection range intermediate between dd-cfDNA values measured in stable subjects and dd-cfDNA values measured in subjects undergoing active rejection, subacute rejection is determined to be occurring.

13. The method of claim 12, wherein
the graft is a graft selected from the group consisting of kidney, heart, lung, liver, skin, cornea, intestine, pancreas, limb, digit, bone, ligament, cartilage, and tendon.

14. The method of claim 13, wherein
the graft is kidney.

15. The method of claim 12, wherein
the sample is a blood sample.

16. The method of claim 12, wherein
the selected dd-cfDNA range indicative of subacute rejection is between 0.5 and 1.5%.

17. The method of claim 12, wherein
the measurement of dd-cfDNA is achieved by a SNP analysis.

18. The method of claim 17, wherein
the SNP analysis encompasses the use of at least 10,000 SNPs.

19. The method of claim 12, wherein
the genotype information of the donor is unknown.

20. The method of claim 12, wherein
the donor and recipient are related.

21. The method of claim 12,
wherein, if the occurrence of a subacute rejection process in the graft recipient is detected, the method comprises the additional steps of:
performing one or more additional diagnostic methods to determine the form of subacute rejection occurring in the graft recipient; and
administering an intervention suitable for the identified form subacute rejection to the graft recipient.

22. A method of assessing graft rejection status in a kidney recipient, comprising the steps of:
measuring dd-cfDNA in the graft recipient by assessment of a sample obtained the kidney recipient;
obtaining a measurement of kidney function by assessment of a sample obtained the kidney recipient;
and
determining kidney transplant status; wherein
if the measured dd-cfDNA value exceeds a selected dd-cfDNA active rejection threshold value, the subject is deemed to be undergoing active rejection;
if the measure dd-cfDNA value is less than the selected dd-cfDNA active rejection threshold value; and if the measure of kidney function is indicative of normal kidney function, the subject is deemed to be stable; and
if the measured dd-cfDNA value is less than the selected dd-cfDNA active rejection threshold value; and if the measure of kidney function is indicative of impaired kidney function, the subject is deemed to be undergoing borderline rejection.

23. The method of claim 22, wherein
the dd-cfDNA threshold active rejection value is a value between 0.75% and 2%.

24. The method of claim 22, wherein
the dd-cfDNA threshold active rejection value is 1%.

25. The method of claim 22, wherein
the dd-cfDNA threshold active rejection value is 1.5%.

26. The method of claim 22, wherein
the measure of kidney function is eGFR score.

27. The method of claim 26, wherein
an eGFR score of 60 or greater is indicative of normal kidney function.

28. The method of claim 22, wherein
the measure of kidney function is creatinine serum level.

29. The method of claim 28, wherein
a creatinine serum value below 1.3 is indicative of normal kidney function.

30. The method of claim 22, wherein
if the kidney recipient is deemed to be undergoing active rejection, the method comprises the additional step of administering to the kidney recipient a suitable intervention for active rejection; and
if the kidney recipient is deemed to be undergoing borderline rejection, the method comprises the additional step of administering to the kidney recipient a suitable intervention for borderline rejection.
